(19) United States
(12) Patent Application Publication
(10) Pub. No.: US 2023/0113656 A1
(43) Pub. Date: Apr. 13, 2023

(54) PATHOLOGICAL CONDITION ANALYSIS SYSTEM, PATHOLOGICAL CONDITION ANALYSIS DEVICE, PATHOLOGICAL CONDITION ANALYSIS METHOD, AND PATHOLOGICAL CONDITION ANALYSIS PROGRAM

(71) Applicant: PST INC., Kanagaw (JP)

(72) Inventors: Yasuhiro OMIYA, Kanagawa (JP); Yorio KUMAMOTO, Tokyo (JP)

(73) Assignee: PST INC., Kanagawa (JP)

(21) Appl. No.: 17/788,150

(22) PCT Filed: Dec. 22, 2020
(86) PCT No.: PCT/JP2020/048056

§ 371 Date: Jun. 22, 2022

(30) Foreign Application Priority Data
Dec. 26, 2019 (JP) 2019-236829

(51) Int. Cl. A61B 5/00; G10L 25/66; G10L 25/21; A61B 5/16

(52) U.S. Cl. CPC A61B 5/4803; G10L 25/66; G10L 25/21; A61B 5/4088; A61B 5/4082; A61B 5/7282; A61B 5/742; A61B 5/165

(57) ABSTRACT

Provided is a pathological condition analysis system using voice, the pathological condition analysis system allowing anyone to perform measurement and estimate a disease anywhere, in a short time, non-invasively, and without being known to others. A pathological condition analysis system according to the present invention analyzes a pathological condition of a subject, and includes: an input means that acquires voice data of the subject; an estimation means that estimates a disease of the subject based on a voice feature amount extracted from the voice data acquired by the input means; and a display means that displays an estimation result by the estimation means, in which the voice feature amount includes the intensity of voice.

### TECHNICAL FIELD

The present invention relates to a pathological condition analysis system, a pathological condition analysis device, a pathological condition analysis method, and a pathological condition analysis program, and particularly relates to a pathological condition analysis system, a pathological condition analysis device, a pathological condition analysis method, and a pathological condition analysis program that analyze a pathological condition using voice.

### BACKGROUND ART

In recent years, there has been disclosed a technique of estimating an emotion or a mental state by analyzing voice by utterance (see Patent Literatures 1 and 2), and it has become possible to measure and quantify a human state by analyzing voice.

In addition, there has also been a scene where voice is actively used for purposes other than communication, for example, a technique for providing a right to access a device by performing personal authentication with a voiceprint (see Patent Literature 3) and a voice recognition technique for operating a machine with voice of a smart home-applicable home appliance or the like (see Patent Literature 4) have been disclosed.

In addition, since each person carries a speech device with spread of a smartphone, utterance can be made at any time as necessary.

Furthermore, if voice is recorded and stored as electronic data, there is an advantage that analysis can be performed retroactively at any time as necessary because the voice does not deteriorate unlike blood or urine.

Meanwhile, a doctor has estimated a disease from a patient's state for a long time. In particular, there is no effective biomarker in a mental/nervous system disease, and therefore a patient's body movement, a patient's way of speaking, a patient's expression, and the like are information sources. For example, it has been empirically known that depression causes a person to speak less, causes a person to speak quietly, and slows a speaking speed, but an index for determining a specific disease has not been reached.

### CITATION LIST

### Patent Literature

### Patent Literature 1: JP 2007-296169 A

### Patent Literature 2: WO 2006/132159 A

### Patent Literature 3: US 2016/0119338 A

### Patent Literature 4: JP 2014-206642 A

### SUMMARY OF INVENTION

### Technical Problem

In order to increase a physical examination ratio leading to prevention and early detection of a disease, there is a demand for a test that can be easily performed by oneself, is inexpensive, and does not require a special opportunity therefor in daily life.

Therefore, one object of the present invention is to provide a pathological condition analysis system, a pathological condition analysis device, a pathological condition analysis method, and a pathological condition analysis program using voice, the pathological condition analysis system, the pathological condition analysis device, the pathological condition analysis method, and the pathological condition analysis program allowing anyone to perform measurement and estimate a disease anywhere, in a short time, non-invasively, and without being known to others.

### Solution to Problem

As a result of intensive studies based on such problems, the present inventors have found that there is a possibility of a specific disease and the severity of the disease can be estimated by using a voice feature amount related to the intensity of sound, and have reached the present invention.

The present invention relates to a pathological condition analysis system that analyzes a pathological condition of a subject, the pathological condition analysis system including: an input means that acquires voice data of the subject; an estimation means that estimates a disease of the subject based on a voice feature amount extracted from the voice data acquired by the input means; and a display means that displays an estimation result by the estimation means, in which the voice feature amount includes the intensity of voice.

### Advantageous Effects of Invention

The present invention can estimate a possibility of a specific disease simply and non-invasively by using a voice feature amount related to the intensity of sound.

In addition, a highly versatile voice feature amount such as the intensity of sound is used, special advanced preprocessing of voice is not required, and a possibility of a specific disease can be estimated with a simple estimation program.

### BRIEF DESCRIPTION OF DRAWINGS

FIG. 1 is a block diagram illustrating a configuration example of an estimation system according to the present invention.

FIG. 2 is a block diagram illustrating a configuration example of the estimation system according to the present invention, which is an example different from that of FIG. 1.

FIG. 3 is a flowchart illustrating an example of estimation processing by an estimation system 100 illustrated in FIG. 1.

FIG. 4 is a table illustrating a calculation result of a voice feature amount.

FIG. 5 illustrates a graph of an ROC curve indicating separation performance between a healthy person or a specific disease and others, and a confusion matrix created at a point where an AUC is obtained and an accuracy ratio is maximized.

FIG. 6 illustrates a graph of an ROC curve indicating separation performance between a healthy person or a specific disease and others, and a confusion matrix created at a point where an AUC is obtained and an accuracy ratio is maximized.

FIG. 7 illustrates a graph of an ROC curve indicating separation performance between a healthy person or a specific disease and others, and a confusion matrix created at a point where an AUC is obtained and an accuracy ratio is maximized.

FIG. 8 is a table illustrating a calculation result of a variation in peak positions.

FIG. 9 is a table illustrating a correlation of BDI and a correlation of HAMD.

FIG. 10 is a graph illustrating a correlation of BDI.

FIG. 11 is a graph illustrating a correlation of BDI.

FIG. 12 is a graph illustrating a correlation of HAMD.

### DESCRIPTION OF EMBODIMENTS

Hereinafter, embodiments of the present invention will be described with reference to the drawings.

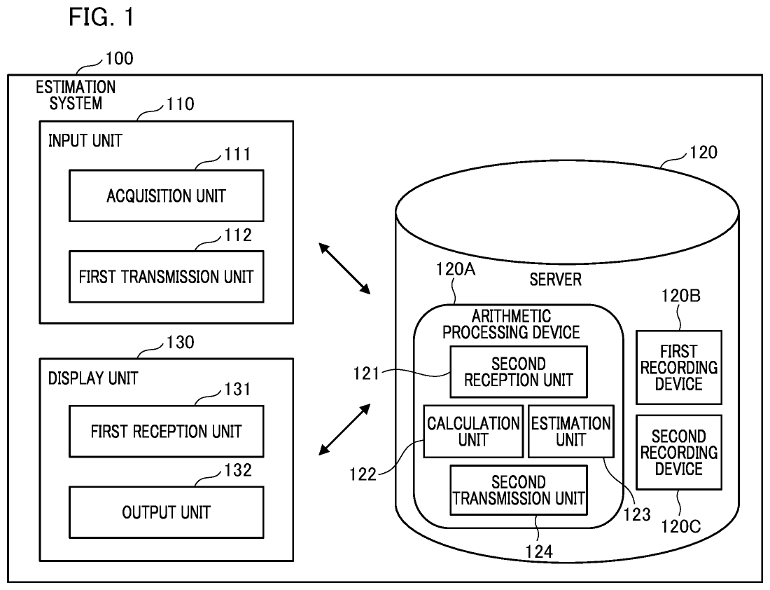

FIG. 1 is a block diagram illustrating a configuration example of an estimation system according to the present invention.

An estimation system 100 in FIG. 1 includes an input unit 110 for acquiring voice of a subject, a display unit 130 for displaying an estimation result and the like to the subject, and a server 120. The server 120 includes an arithmetic processing device 120A (for example, a CPU), a first recording device 120B such as a hard disk in which an estimation program that is a program executed by the arithmetic processing device 120A is recorded, and a second recording device 120C such as a hard disk in which physical examination data of the subject and a collection of messages to be transmitted to the subject are recorded. The server 120 is connected to the input unit 110 and the display unit 130 in a wired or wireless manner. The arithmetic processing device 120A may be implemented by software or hardware.

The input unit 110 includes a voice acquisition unit 111 such as a microphone and a first transmission unit 112 that transmits acquired voice data to the server 120. The acquisition unit 111 generates voice data of a digital signal from an analog signal of voice of the subject. The voice data is transmitted from the first transmission unit 112 to the server 120.

The input unit 110 acquires a voice signal uttered by the subject via the voice acquisition unit 111 such as a microphone, and samples the voice signal at a predetermined sampling frequency (for example, 11025 Hz) to generate voice data of a digital signal.

The input unit 110 may include a recording unit that records voice data separately from the recording device on the server 120 side. In this case, the input unit 110 may be a portable recorder. The recording unit of the input unit 110 may be a recording medium such as a CD, a DVD, a USB memory, an SD card, or a mini disk.

The display unit 130 includes a first reception unit 131 that receives data such as an estimation result and an output unit 132 that displays the data. The output unit 132 is a display that displays data such as an estimation result. The display may be an organic electro-luminescence (EL), a liquid crystal, or the like.

Note that the input unit 110 may have a function such as a touch panel in order to input data of a result of a medical examination or data of an answer regarding a stress check in advance. In this case, the input unit 110 and the display unit 130 may be implemented by the same hardware having functions of the input unit 110 and the display unit 130.

The arithmetic processing device 120A includes a second reception unit 121 that receives voice data transmitted from the first transmission unit 112, a calculation unit 122 that calculates a prediction value of a disease based on a voice feature amount related to the intensity of sound extracted from voice data of the subject, an estimation unit 123 that estimates a disease of the subject using the prediction value of a disease as an input, and a second transmission unit 124 that transmits data related to an estimation result and the like to the display unit 130. Note that the calculation unit 122 and the estimation unit 123 have been separately described in order to describe the functions, but the functions of the calculation unit and the estimation unit may be performed simultaneously. In addition, the calculation unit and the estimation unit can also estimate a disease by creating a learned model by machine learning using learning data and inputting data (test data) of the subject to the learned model. However, in the present invention, since the voice feature amount including the intensity of voice used for estimation of a disease can be calculated by an ordinary computer, it is not always necessary to use machine learning. Note that, in the present specification, the term “mental value” is used synonymously with the prediction value of a disease.

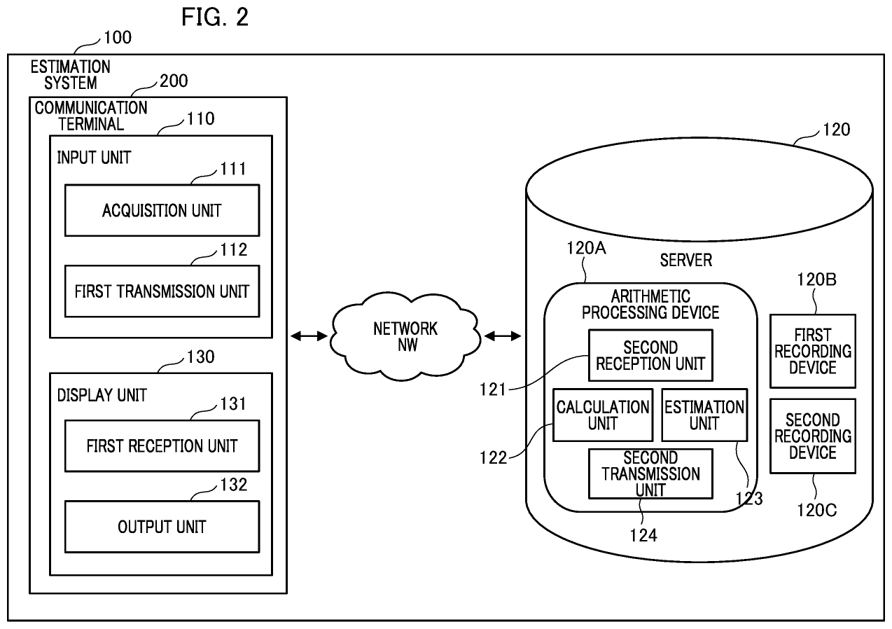

FIG. 2 is a block diagram illustrating a configuration example of the estimation system according to the present invention, which is an example different from that of FIG. 1.

An estimation system 100 in FIG. 2 connects the server 120 of the estimation system 100 in FIG. 1 to the input unit 110 and the display unit 130 via a network NW. In this case, the input unit 110 and the display unit 130 are communication terminals 200. The communication terminal 200 is, for example, a smartphone, a tablet-type terminal, or a notebook personal computer or a desktop personal computer including a microphone.

The network NW connects the communication terminal 200 and the server 120 to each other via a mobile phone communication network or a wireless LAN based on a communication standard such as wireless fidelity (Wi-Fi) (registered trademark). The estimation system 100 may connect a plurality of the communication terminals 200 and the server 120 to each other via the network NW.

The estimation system 100 may be implemented by the communication terminal 200. In this case, an estimation program stored in the server 120 is downloaded to the communication terminal 200 via the network NW and recorded in a recording device of the communication terminal 200. A CPU included in the communication terminal 200 executes the estimation program recorded in the recording device of the communication terminal 200, whereby the communication terminal 200 may function as the calculation unit 122 and the estimation unit 123. The estimation program may be distributed by being recorded in an optical disk such as a DVD or a portable recording medium such as a USB memory.

### First Embodiment

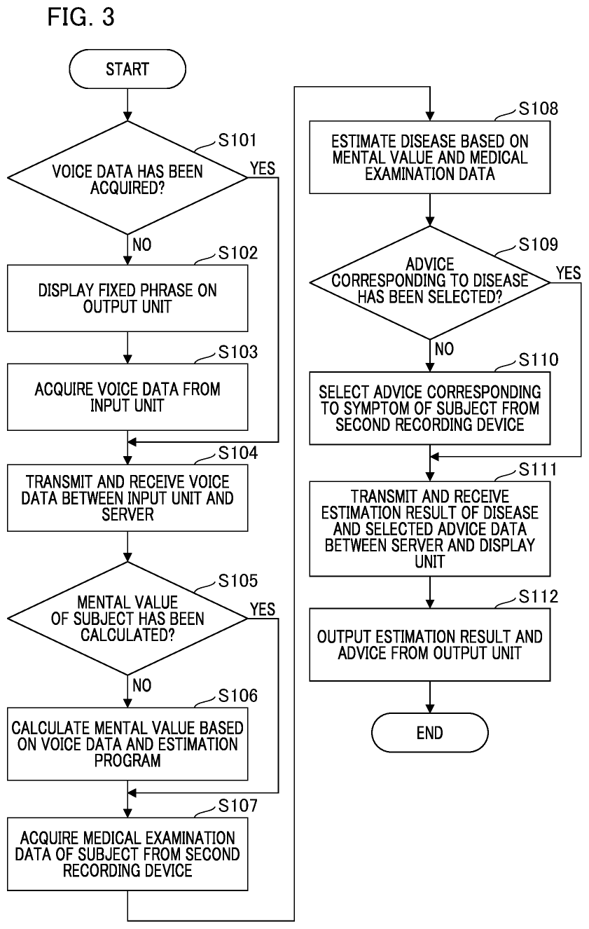

FIG. 3 is a flowchart illustrating an example of estimation processing by the estimation system 100 illustrated in FIG. 1.

The processing illustrated in FIG. 3 is implemented by execution of an estimation program recorded in the first recording device 120B by the arithmetic processing device 120A in the estimation system 100. Each of functions of the second reception unit 121, the calculation unit 122, the estimation unit 123, and the second transmission unit 124 of the arithmetic processing device 120A will be described with reference to FIG. 3.

(Calculation Unit 122)

When the processing is started, in step S101, the calculation unit 122 determines whether or not voice data has been acquired by the acquisition unit 111. When the voice data has already been acquired, the process proceeds to step S104. When the voice data has not been acquired, the calculation unit 122 commands the output unit 132 of the display unit 130 to display a predetermined fixed phrase in step S102.

The present estimation program does not estimate a mental/nervous system disease according to the meaning or content of utterance of a subject. Therefore, the voice data acquired by the acquisition unit 111 may be any voice data as long as the voice data has a total utterance time of about two to 300 seconds. A language used is not particularly limited, but is desirably the same as a language used by a population at the time of creating the estimation program. Therefore, the fixed phrase displayed on the output unit 132 may be any fixed phrase as long as the fixed phrase uses the same language as the population and has a total utterance time of about two to 300 seconds. Preferably, the fixed phrase desirably has a total utterance time of about three to 180 seconds.

For example, the fixed phrase may be “Irohanihoheto”, “Aiueokakikukoko, or the like including no special emotion, or may be a response to a question such as “What is your name?” or “When is your birthday?”.

Among these, words including “ga”, “gi”, “gu”, “ge”, and “go” which are voiced sounds (palatal sounds), “pa”, “pi”, “pu”, “pe”, and “po” which are semi-voiced sounds (lip sounds), and “ra”, “ri”, “ru”, “re”, and “ro” which are lingual sounds are preferably used. Repetition of “pataka” is more preferable. Typically, a word of repeating “pataka” for three to ten seconds or about five to ten times is used.

A voice acquisition environment is not particularly limited as long as the voice acquisition environment is an environment in which only voice uttered by the subject can be acquired, but is preferably an environment of 40 bB or less. Voice uttered by the subject is more preferably acquired in an environment of 30 dB or less.

When the subject reads the fixed phrase, in step S103, the calculation unit 122 acquires voice data from the voice uttered by the subject, and the process proceeds to step S104.

Next, in step S104, the calculation unit 122 commands the input unit 110 to transmit the voice data to the second reception unit 121 of the server 120 via the first transmission unit 112.

Next, in step S105, the calculation unit 122 determines whether or not a mental value of the subject, that is, a prediction value of a disease of the subject has been calculated. In the present invention, the prediction value of a disease is a feature amount F(a) including a combination of voice feature amounts generated by extracting one or more acoustic parameters, and is a prediction value of a specific disease. The acoustic parameter is obtained by parameterizing a feature when sound is transmitted. When the prediction value of a disease has already been calculated, the process proceeds to step S107. When the prediction value of a disease has not been calculated, in step S106, the calculation unit 122 calculates the prediction value of a disease based on the voice data of the subject and the estimation program.

In step S107, the calculation unit 122 acquires medical examination data of the subject acquired in advance from the second recording device 120C. Note that the arithmetic processing device 120A may omit step S107 and estimate a disease from the prediction value of a disease without acquiring the medical examination data.

Next, in step S108, the estimation unit 123 estimates a disease by the prediction value of a disease calculated by the calculation unit 122 alone or by combining the prediction value of a disease and the medical examination data.

The estimation unit 123 can discriminate a plurality of patients for whom prediction values of a disease have been calculated into a target to be specified and the others by providing individual thresholds for distinguishing a specific disease from the others regarding the prediction value of a disease. In Examples described later, determination is made by classifying the prediction values of a disease into a case where the prediction value exceeds a threshold and a case where the prediction value does not exceed the threshold.

Next, in step S109, the estimation unit 123 determines whether or not advice data corresponding to a disease has been selected. The advice data corresponding to a disease is advice for preventing a disease or avoiding exacerbation of a disease when the subject receives the advice data. When the advice data has been selected, the process proceeds to step S111.

When the advice data has not been selected, in step S110, the estimation unit 123 selects advice data corresponding to the symptom of the subject from the second recording device 120C.

Next, in step S111, the estimation unit 123 gives a command to transmit the estimation result of a disease and the selected advice data to the first reception unit 131 of the display unit 130 via the second transmission unit 124.

Next, in step S112, the estimation unit 123 commands the output unit 132 of the display unit 130 to output the estimation result and the advice data. Finally, the estimation system 100 ends the estimation processing.

### Second Embodiment

1. Pathological Condition Analysis Method

(1) Utterance and Acquisition of Voice Thereof

In the present invention, the type (phrase) of the utterance acquired by the acquisition unit 111 in FIG. 1 is not particularly limited. However, since the voice feature amount related to the intensity of sound is used, repetition of several sounds is preferable because analysis is easier.

Examples of the repetition of some sounds include “Aiueo Aiueo . . . ”, “Irohanihoheto Irohanihoheto . . . ”, and “PatakaPatakaPataka . . . ”.

These phrases are uttered by the subject by being repeated usually about three to ten times, preferably about four to six times, or usually about two to 20 seconds, preferably about three to seven seconds.

The voice thus uttered is recorded by a recorder or the like as the acquisition unit 111.

(2) Normalization of Volume

Volume normalization is one type of acoustic signal processing, and is processing of analyzing the volume (program level) of entire certain voice data and adjusting the volume to a specific volume. Volume normalization is used for the purpose of adjusting the voice data to an appropriate volume and unifying the volumes of a plurality of pieces of voice data.

(3) Calculation of Intensity of Sound

Voice is displayed as a waveform (obtained by measuring a sound pressure as a voltage value). In order to obtain the intensity of sound (how much a waveform fluctuates), processing such as taking an absolute value or taking a square is performed to convert the sound into a positive numerical value.

(4) Detection of Peak Position

In a graph of the intensity of sound, a peak threshold is set, and a peak position is detected.

(5) A voice feature amount related to a peak position (that is, the intensity of voice) is extracted. Examples thereof include the following voice feature amounts.

A: Slope of peak value linear approximation for each phoneme

B: Average value of peak positions for each phoneme

C: Variation in peak positions for each phoneme

D: Slope of peak position linear approximation throughout voice

E: Average value of peak intervals for each phoneme

F: Variation in peak intervals for each phoneme

Here, for example, in a case of repetition of “pataka”, the phoneme refers to a pronunciation of each of “pa”, “ta”, and “ka”.

(6) Whether or not there is a significant difference in the voice feature amount is verified based on voice of a patient of each disease.

### Example 1

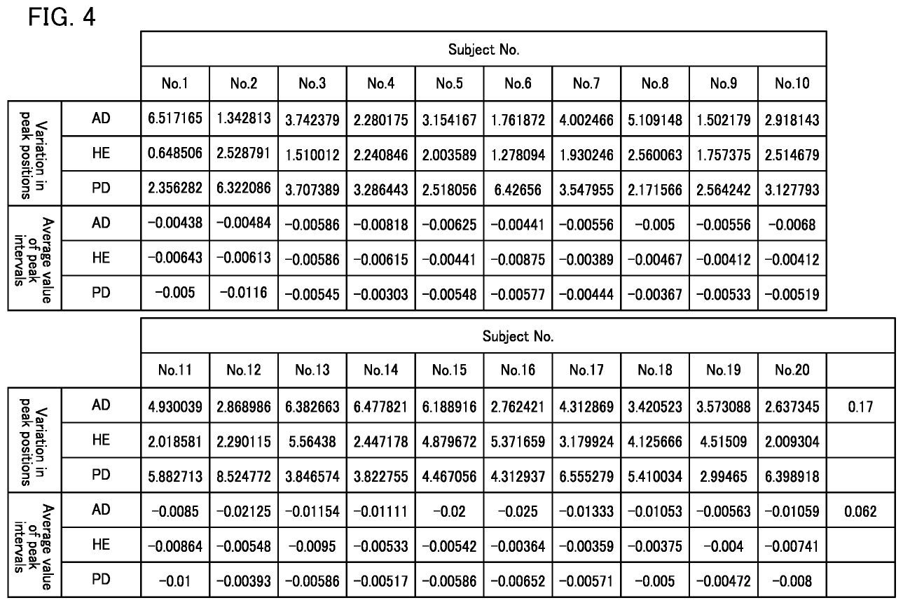

Twenty Alzheimer's dementia patients (represented by AD in the drawings), 20 Parkinson's disease patients (represented by PD in the drawings), and 20 healthy persons (represented by HE in the drawings) are used as subjects. Voice feature amounts were calculated for voice of these subjects (obtained by repeatedly uttering “pataka” about five times). FIG. 4 is a table illustrating a calculation result of the voice feature amounts.

As a result of analyzing the voice feature amount regarding the intensity of sound indicated by A to F in the above (5), regarding the “variation in peak positions”, a significant difference was recognized in the voice feature amount between the Parkinson's disease patients (PD) and the healthy persons (HE).

In addition, regarding the “average value of peak intervals”, a significant difference was recognized in the voice feature amount between Alzheimer's dementia patients (AD) and healthy persons (HE), and a significant difference was recognized in the voice feature amount between Alzheimer's dementia patients (AD) and Parkinson's disease patients (PD).

Based on the above calculation result, an ROC curve was drawn as an evaluation index of machine learning, and an AUC was obtained. ROC is an abbreviation of Receiver Operating Characteristic. AUC is an abbreviation of Area under the ROC curve.

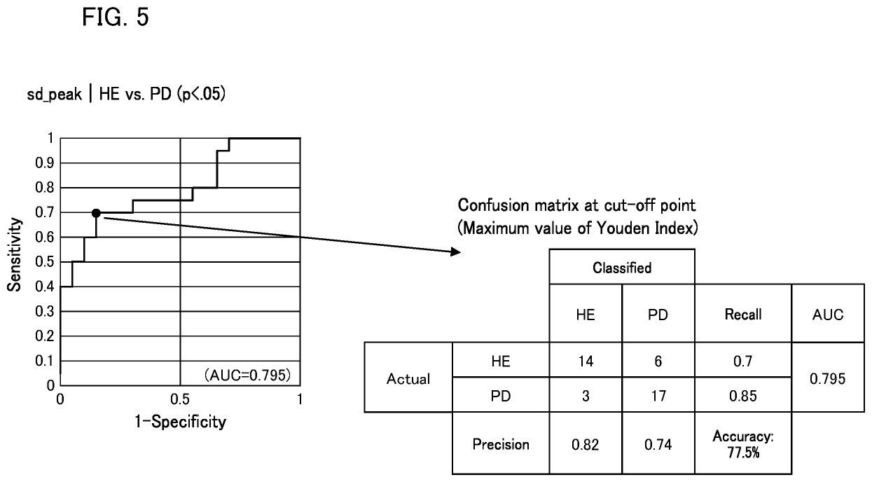

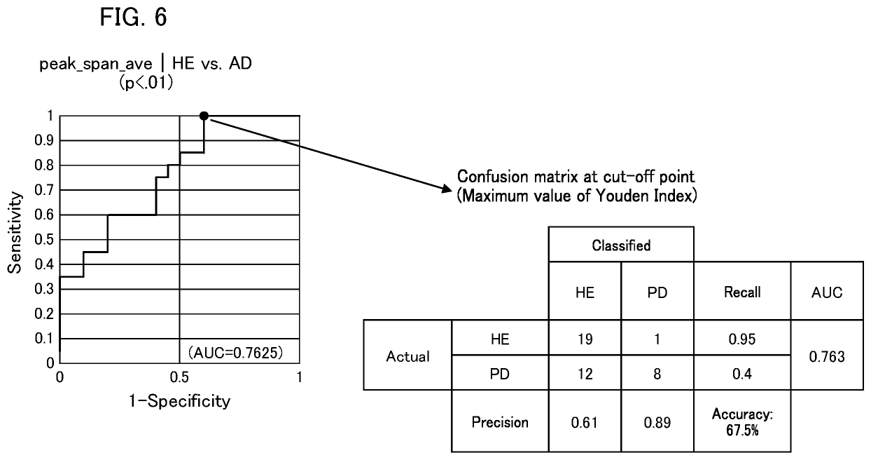

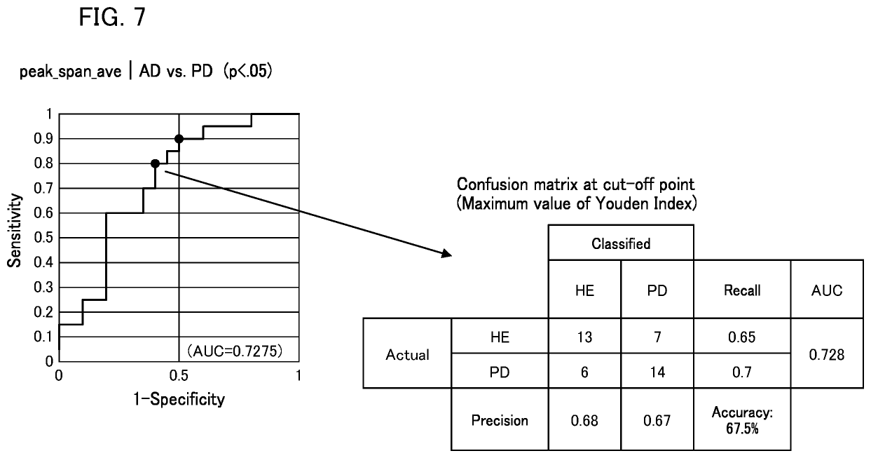

FIGS. 5, 6, and 7 each illustrate a graph of an ROC curve indicating separation performance between a healthy person or a specific disease and others, and a confusion matrix created at a point where an AUC is obtained and an accuracy ratio is maximized. FIG. 5 illustrates healthy persons and Parkinson's disease patients, FIG. 6 illustrates healthy persons and Alzheimer's dementia patients, and FIG. 7 illustrates Alzheimer's dementia patients and Parkinson's disease patients. In FIGS. 5, 6, and 7, the horizontal axis represents 1−specificity, and the vertical axis represents sensitivity.

### Example 2

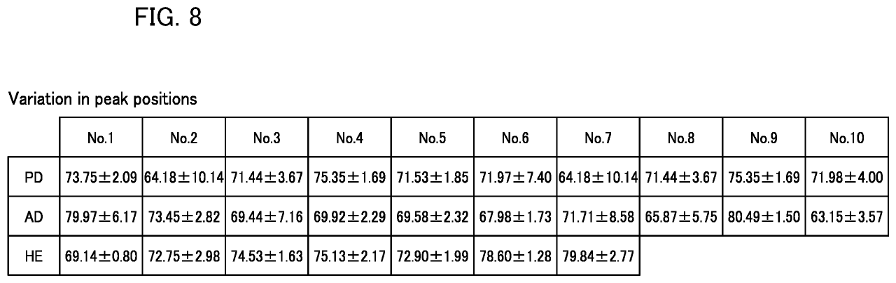

For voice of ten Alzheimer's dementia patients, seven Parkinson's disease patients, and seven healthy persons (obtained by repeatedly uttering “pataka” about five times) recorded in the same facility, peak positions were detected, and a variation in peak positions was calculated. The calculation result is illustrated in FIG. 8. FIG. 8 is a table illustrating the calculation result of the variation in peak positions.

### Example 3

A correlation between BDI widely used as a test index of depression and a voice feature amount was verified. BDI is an abbreviation of Beck Depression Inventory. In addition, a correlation between HAMD and a voice feature amount was verified. HAMD is an abbreviation of Hamilton Depression Rating Scale.

Method:
Voice (96 kHz, 24 bits, wav file) data was collected from major depressive disorder patients. The data was collected from 9 male and 14 female participants (mean age: 31.6±7.0; 19-41 years) using a portable recorder and a pin microphone. As for the voice of the participants, the participants repeatedly uttered “pataka” for about 5 seconds. Furthermore, a psychological test of “Hamilton depression rating scale” (HAMD-21) and “Beck depression inventory” (BDI) was performed before recording was started. Recording and psychological test results were collected at the first visit and when a symptom was halved (a symptom was reduced by half).
The above six features related to the intensity of recorded voice were examined based on consideration that depression affects the intensity of voice. Next, correlation analysis between the intensity data and the result of the psychological test was examined.

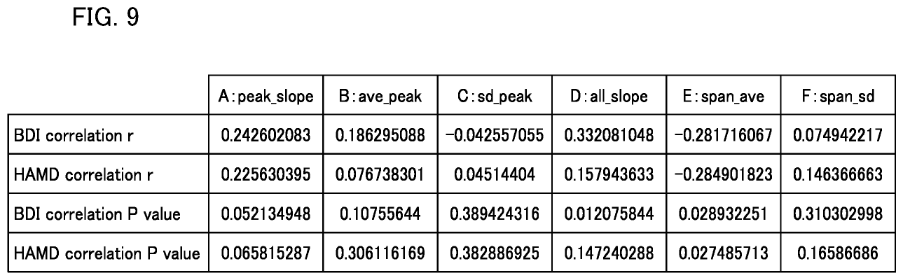

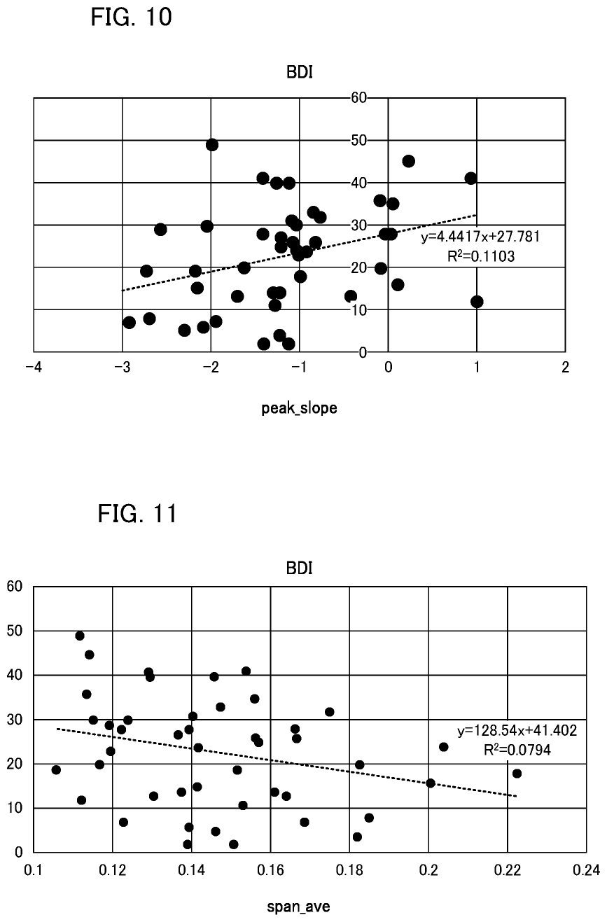

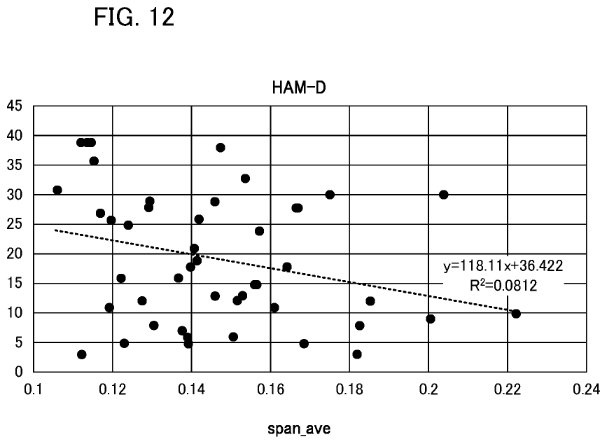

FIG. 9 is a table illustrating a correlation of BDI and a correlation of HAMD. FIGS. 10 and 11 are graphs illustrating a correlation of BDI. FIG. 12 is a graph illustrating a correlation of HAMD.

Results:
Analysis of the scores of the psychological test with the six features has revealed a correlation of the following three combinations.
There is a significant correlation between the uttered “slope of peak position linear approximation throughout voice” and the BDI score.
That is, as the BDI score is higher (as the symptom of depression is severer), voice tends to become larger as the utterance progresses more than voice immediately after the start.
In addition, the “average value of peak positions for each phoneme” has a significant correlation with the BDI score and the HAMD 21 score.

This indicates that the degree of depression symptom can be estimated by performing analysis using the voice feature amount including the intensity of voice.

[Implementation by Software]

Each processing illustrated in FIG. 3 may be implemented by a logic circuit (hardware) formed in an integrated circuit (IC chip) or the like, or may be implemented by software using a central processing unit (CPU).

When each processing illustrated in FIG. 3 is implemented by software, a user client terminal 100, a skin disease analysis device 200, and an administrator client terminal 300 each include a CPU that executes a command of a program which is software that implements each function, a read only memory (ROM) or a storage device (referred to as a “recording medium”) in which the program and various types of data are recorded so as to be readable by a computer (or CPU), a random access memory (RAM) in which the program is developed, and the like. Then, the computer (or CPU) reads the program from the recording medium and executes the program, thereby achieving the object of the present invention. As the recording medium, a “non-transitory tangible medium”, for example, a tape, a disk, a card, a semiconductor memory, a programmable logic circuit, or the like can be used. In addition, the program may be supplied to the computer via an arbitrary transmission medium (communication network, broadcast wave, or the like) capable of transmitting the program. Note that one aspect of the present invention can also be implemented in a form of a data signal embedded in a carrier wave in which the program is embodied by electronic transmission.

The present invention is not limited to the above-described embodiments, and various modifications can be made within the disclosed scope. Embodiments obtained by appropriately combining technical means disclosed in the different embodiments are also included in the technical scope of the present invention.

The present application claims priority based on Japanese Patent Application No. 2019-236829 filed on Dec. 26, 2019, the entire contents of which are incorporated herein by reference.

### REFERENCE SIGNS LIST

100 Estimation system
110 Input unit
120 Server
130 Display unit

1. A pathological condition analysis system that analyzes a pathological condition of a subject, the pathological condition analysis system comprising:
an inputter that acquires voice data of the subject;
an estimator that estimates a disease of the subject based on a voice feature amount extracted from the voice data acquired by the inputter; and
a display that displays an estimation result by the estimator, wherein
the voice feature amount includes an intensity of voice.

2. The pathological condition analysis system according to claim 1, wherein
the disease estimated by the estimator includes Alzheimer's dementia and Parkinson's disease.

3. The pathological condition analysis system according to claim 1, wherein the estimator estimates whether the disease is Parkinson's disease or Alzheimer's dementia.

4. The pathological condition analysis system according to claim 1, wherein the feature amount including the intensity of the voice is a variation in peak positions or an average value of peak intervals.

5. The pathological condition analysis system according to claim 1, wherein the estimator estimates a degree of depression symptom.

6. The pathological condition analysis system according to claim 1, wherein the feature amount including the intensity of the voice is a slope of peak position linear approximation throughout the voice or an average value of peak positions.

7. The pathological condition analysis system according to claim 1, wherein the voice data is utterance of a word including a palatal sound, a lip sound, or a lingual sound.

8. The pathological condition analysis system according to claim 7, wherein the voice data is utterance of repetition of “pataka”.

9. A pathological condition analysis device that analyzes a pathological condition of a subject, the pathological condition analysis device comprising:
an input unit that acquires voice data of the subject;
an estimation unit that estimates a disease of the subject based on a voice feature amount extracted from the voice data acquired by the input unit; and
a display unit that displays an estimation result by the estimation unit, wherein
the voice feature amount includes an intensity of voice.

10. A pathological condition analysis method executed by a pathological condition analysis system that analyzes a pathological condition of a subject, the pathological condition analysis method comprising:
acquiring voice data of the subject;
estimating a disease of the subject by machine learning using a prediction value of a disease based on a voice feature amount extracted from the voice data acquired as an input; and
displaying an estimation result of the estimating, wherein
the voice feature amount includes an intensity of voice.

11. A non-transitory computer-readable storage medium storing a program for causing a computer to function as the inputter, estimator and display of the pathological condition analysis system according to claim 1.
